(19) United States
(12) Patent Application Publication
(10) Pub. No.: US 2010/0273242 A1
(43) Pub. Date: Oct. 28, 2010

(54) BIOLOGICAL DESULFURIZATION APPARATUS

(76) Inventors: Takumi Obara, Fuchu-shi (JP); Yasuhiko Nagamori, Kawasaki-shi (JP); Nobuyuki Ashikaga, Tokyo (JP); Hiroshi Tamura, Tokyo (JP); Takayuki Ishige, Yokohama-shi (JP); Takeo Yamamori, Kawasaki-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/527,549

(22) PCT Filed: Dec. 16, 2008
(86) PCT No.: PCT/JP2008/073266

§ 371 Date: Aug. 17, 2009

(30) Foreign Application Priority Data
Jan. 16, 2008 (JP) 2008-007188

(51) Int. Cl. C02F 3/34; C12M 1/00

(52) U.S. Cl. 435/262; 435/289.1

(57) ABSTRACT

Disclosed is a desulfurization apparatus including a biological reaction tank 1 into which a hydrogen sulfide gas-containing gas is introduced, a carrier-packed layer 2a, 2b that is arranged in the biological reaction tank 1 and packed with a carrier for adhesion of microorganisms, a means that feeds an oxygen-containing gas to the biological reaction tank 1, and two or more sprinkling mechanisms 3, 4 that sprinkle water necessary for the organisms on the upper part of the biological reaction tank 1.

### TECHNICAL FIELD

The present invention relates to a biological desulfurization apparatus for biogas generated particularly by anaerobic digestive treatment of organic drainage such as sewage and industrial drainage.

### BACKGROUND ART

A methane fermentation process is often used as a method of treating sewage sludge, organic wastes such as raw garbage, and organic drainage such as food factory effluent. The methane fermentation process is a treatment process wherein organic wastes or organic drainage is introduced into a biological reaction tank to allow a group of methane fermentation bacteria in the biological reaction tank to decompose organic matters, thereby forming a biogas consisting primarily of methane gas and simultaneously decomposing and removing organic matters in the drainage. However, when sulfur components such as those derived from proteins are contained in drainage, the sulfur components are reduced by the action of sulfate reducing bacteria, and thus hydrogen sulfide gas is formed in the biogas.

When methane gas contained in biogas is used as a fuel in a boiler, an electric generator etc., hydrogen sulfide gas contained in biogas should be removed. This is because the hydrogen sulfide gas in biogas is oxidized upon combustion of biogas, to form sulfur oxides which can corrode facilities.

The method used in removing the hydrogen sulfide gas contained in biogas includes a dry-desulfurization method of removing the gas by adsorption onto an adsorbent consisting primarily of iron oxide and a wet-desulfurization method of removing the gas by absorption into an aqueous solution using an alkali etc. However, these methods are in a system where running costs are rocketed because chemicals such as an adsorbent are required for adsorption and the adsorbent after adsorption turns to waste.

Accordingly, the inventors have previously proposed, as a system of desulfurization at low running cost, a technique of removing hydrogen sulfide in biogas by filling a reaction tank with a packing material to which a microorganism decomposing hydrogen sulfide by oxidation has adhered (Japanese Patent Application No. 2007-20018). This technique makes use of an apparatus wherein treated water subjected to aerobic treatment is allowed to flow downward in a biological reaction tank filled with a packing material having microorganism adhered thereto, thereby feeding water and alkalinity necessary for the microorganisms, while hydrogen sulfide-containing biogas and air are introduced upward, thereby treating and oxidatively removing hydrogen sulfide in the gas.

This previous invention relates to a biological desulfurization apparatus including a biological reaction tank packed with a packing material for adhesion of microorganisms, into which a hydrogen sulfide gas-containing gas is introduced, a means that feeds an oxygen-containing gas to the biological reaction tank, and a sprinkling means that sprinkles water (for example, treated water after biological treatment) necessary for the microorganisms on the upper part of the biological reaction tank. In starting up the apparatus, however, there are the following problems:

(1) Unless the biological desulfurization apparatus is rapidly started up, effective utilization of biogas in a boiler or in an electric generator is infeasible because of problems such as corrosion attributable to the concentration of hydrogen sulfide in the gas.

(2) Rapid start-up and stable treatment after start-up are hardly simultaneously achieved.

(3) The sprinkling unit is clogged.

To solve these problems, it is important that sulfur-oxidizing bacteria are allowed to adhere to a packing material, and also that whether or not the amount of the sulfur-oxidizing bacteria that have adhered to the packing material is sufficient for achieving the intended removal of hydrogen sulfide is judged with some sort of indicator.

### DISCLOSURE OF INVENTION

An object of the present invention is to provide a biological desulfurization apparatus which can be started up by adhesion of sulfur-oxidizing bacteria to a carrier-packed layer under circulation of microorganism-containing liquid or sludge and can judge the timing of switching from circulation of microorganism-containing liquid or sludge to water sprinkling, as well as a method of starting up the same.

The biological desulfurization apparatus in one aspect of the invention comprises a biological reaction tank into which a hydrogen sulfide gas-containing gas is introduced, a carrier-packed layer that is arranged in the biological reaction tank and packed with a carrier for adhesion of microorganisms, a means that feeds an oxygen-containing gas to the biological reaction tank, and two or more sprinkling mechanisms that sprinkle water necessary for the organisms onto the upper part of the biological reaction tank.

According to the present invention, there can be provided a biological desulfurization apparatus which can be started up by adhesion of sulfur-oxidizing bacteria to a carrier-packed layer under circulation of microorganism-containing liquid or sludge and can judge the timing of switching from circulation of microorganism-containing liquid or sludge to water sprinkling, as well as a method of starting up the same.

### BRIEF DESCRIPTION OF DRAWINGS

FIG. 1A is a schematic overall view of the biological desulfurization apparatus of the invention in Example 1.

FIG. 1B is a bottom view of a pipe in a first sprinkling mechanism in FIG. 1A.

FIG. 2 is a schematic overall view of the biological desulfurization apparatus of the invention in Example 2.

FIG. 3 is a schematic overall view of the biological desulfurization apparatus of the invention in Example 3.

FIG. 4 is a schematic overall view of the biological desulfurization apparatus of the invention in Example 4.

FIG. 5 is a schematic overall view of the biological desulfurization apparatus of the invention in Example 5.

### BEST MODE FOR CARRYING OUT THE INVENTION

Hereinafter, the biological desulfurization apparatus according to the present invention is described in detail.

(1) The biological desulfurization apparatus of the present invention, as described above, comprises a biological reaction tank, a carrier-packed layer, a means that feeds an oxygen-containing gas to the biological reaction tank, and two or more sprinkling mechanisms. The sprinkling mechanisms are provided with a first sprinkling mechanism for introducing a microorganism-containing liquid or sludge as seed sludge for biological desulfurization and circulating it through the biological reaction tank and a second sprinkling mechanism for spraying water having few solids (biologically treated water) on the carrier-packed layer in the biological reaction tank. The first sprinkling mechanism is provided with a circulation tank, a circulation pump and a pipe (circulation pipe) that connects them for introducing and circulating, as seed sludge for biological desulfurization, a microorganism-containing liquid or sludge for example to the biological reaction tank.

(2) The method of starting up a biological desulfurization apparatus according to the present invention is a method of starting up the biological desulfurization apparatus of the above-mentioned (1), which comprises starting up the biological desulfurization apparatus while microorganisms are allowed to adhere to a carrier by feeding a hydrogen sulfide-containing gas and an oxygen-containing gas to a biological reaction tank under circulation of a microorganism-containing liquid or sludge, wherein the biological desulfurization apparatus has first and second sprinkling mechanisms, wherein the first sprinkling mechanism is provided with a circulation tank, a circulation pump and a pipe for introducing a microorganism-containing liquid or sludge as seed sludge for biological desulfurization and circulating it through the biological reaction tank, and in the first sprinkling mechanism, the pipe for connecting the circulation tank to the biological reaction tank extends to the inside of the biological reaction tank, and the extending portion of the pipe is provided with a hole of 5 to 20 mm in diameter, and the second sprinkling mechanism is arranged on the top of the biological reaction tank and provided with a pipe for sprinkling water on the carrier-packed layer, and the pipe in the second sprinkling mechanism extends to the inside of the biological reaction tank, and the extending portion of the pipe is provided with a nozzle excellent in an ability to disperse water.

The method of switching to sprinkling water on the upper part of the biological reaction tank includes:

(2-1) The method wherein the pH and/or alkalinity of discharged water from the biological reaction tank is measured, and when the pH and/or alkalinity of the discharged water from the biological reaction tank is reduced, circulating the microorganism-containing liquid or sludge is stopped and switched to sprinkling water on the upper part of the biological reaction tank.

(2-2) The method wherein the concentration of hydrogen sulfide gas in gas at the outlet of the biological reaction tank is measured, and when the concentration of hydrogen sulfide in gas at the outlet is reduced, circulating the microorganism-containing liquid or sludge is stopped and switched to sprinkling water on the upper part of the biological reaction tank.

(2-3) The method wherein the turbidity of water in the circulation tank is detected, and when turbidity in the circulation tank is confirmed, circulating the microorganism-containing liquid or sludge is stopped and switched to sprinkling water on the upper part of the biological reaction tank.

(2-4) A method which comprises switching from circulating the microorganism-containing liquid or sludge to sprinkling water on the upper part of the biological reaction tank by judging, by two or more of the methods of the above-mentioned (2-1) to (2-3), the timing of the switching.

In the present invention, a digestive liquid from facilities where anaerobic digestive treatment is conducted, or digestive sludge, can be used as the seed sludge.

Now, specific examples of the biological desulfurization apparatus in accordance with embodiments of the invention are described in detail with reference to the drawings. However, the embodiments of the invention are not limited to the following description.

### EXAMPLE 1

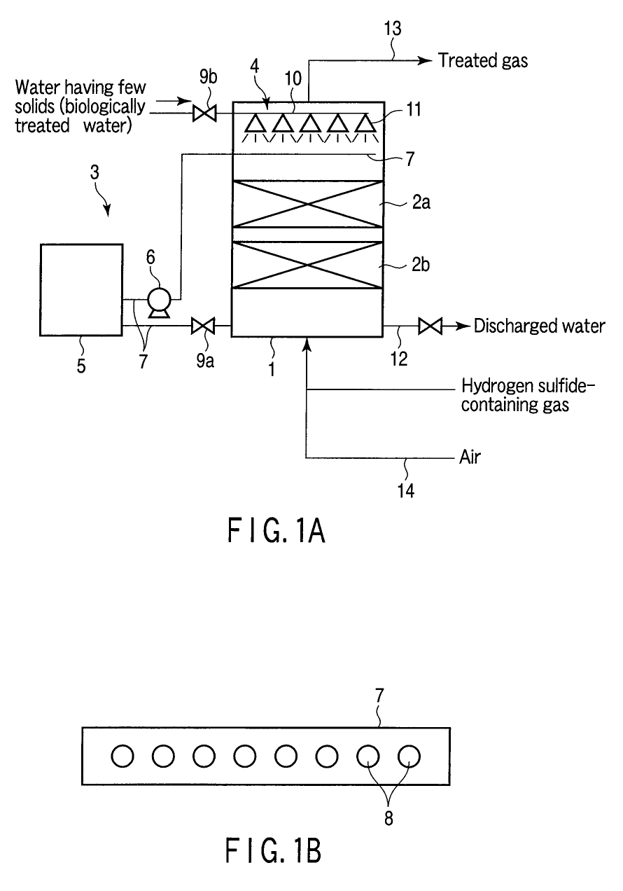

The biological desulfurization apparatus of the invention in Example 1 is described with reference to FIG. 1A and FIG. 1B. FIG. 1A is a schematic overall view of the biological desulfurization apparatus, and FIG. 1B is a bottom view of a pipe in a first sprinkling mechanism in FIG. 1A.

In the figure, 1 is a biological reaction tank into which a hydrogen sulfide gas-containing gas is introduced. Carrier-packed layers 2a, 2b each filled with a carrier for adhesion of microorganisms are arranged vertically in the biological reaction tank 1. The biological reaction tank 1 is supplied with a hydrogen sulfide-containing gas and air from the bottom. A first sprinkling mechanism 3 and a second sprinkling mechanism 4 that sprinkle water necessary for organisms on an upper part in the biological reaction tank 1 are arranged in the biological reaction tank 1 and its vicinity respectively.

The first sprinkling mechanism 3 is one wherein a microorganism-containing liquid or sludge is introduced as seed sludge for biological desulfurization into, and circulated through, the biological reaction tank 1. The first sprinkling mechanism 3 is provided with a circulation tank 5, a circulation pump 6, and circulation pipes 7 for connecting the circulation tank 5 to the biological reaction tank 1. The circulation pipe 7 extends to the inside of the biological reaction tank 1, and the extending portion of this pipe is provided with a plurality of holes 8 of 5 to 20 mm in diameter (see FIG. 1A). The hole 8 is to spray a microorganism-containing liquid or sludge on the carrier-packed layers 2a, 2b and is configured to be larger than the hole diameter of a shower nozzle described later. The circulation pipe 7 has a valve 9a interposed therein.

The second sprinkling mechanism 4 is provided with a pipe 10 extending to the inside of the biological reaction tank. Shower nozzles 11 excellent in the ability to disperse water are arranged at the extending portion of the pipe 10. A valve 9b is interposed in the pipe 10. From the shower nozzles 11 of the pipe 10, water having few solids (for example, treated water resulting from biological treatment of discharged water; referred to hereinafter as biologically treated water) is sprayed on the carrier-packed layers 2a, 2b. In the figure, the numeral 12 refers to a drain pipe, the numeral 13 to a treated-gas pipe, and the numeral 14 to an air feeding pipe for feeding air (a means of feeding an oxygen-containing gas) to the biological reaction tank 1.

The biological desulfurization apparatus in Example 1, as shown in FIG. 1, is provided with a biological reaction tank 1 into which a hydrogen sulfide gas-containing gas is introduced, carrier-packed layers 2a, 2b that are arranged in the biological reaction tank and packed with a carrier for adhesion of microorganisms, a means that feeds an oxygen-containing gas to the biological reaction tank, a first sprinkling mechanism 3 that introduces a microorganism-containing liquid or sludge to the upper part in the biological reaction tank, and a second sprinkling mechanism 4 that sprays biologically treated water on the upper part in the biological reaction tank.

When the biological desulfurization apparatus thus constituted is started up, the circulation tank 5 into which a microorganism-containing liquid or sludge has been introduced, the circulation pump 6 and the circulation pipe 7 are used to circulate and feed the microorganism-containing liquid or sludge to the biological reaction tank 1, while a hydrogen sulfide-containing gas and air are fed to the biological reaction tank and microorganisms are allowed to adhere to the carriers of the carrier-packed layer 2a, 2b. By such start-up, sulfur-oxidizing bacteria are allowed to adhere to the packed layers 2a, 2b and used as seed bacteria to be proliferated as sulfur-oxidizing bacteria adhering to the packed layers 2a, 2b.

In FIG. 1, if a liquid containing a large amount of solids, such as digestive sludge, were circulated though the same pipe as that having shower nozzles for sprinkling biologically treated water, clogging of the shower nozzles with the solids would be worried about. Even if the raw liquid to be circulated is a liquid with few solids, solid element sulfur can be partially formed upon oxidation of hydrogen sulfide in gas by the action of sulfur-oxidizing bacteria, to cause clogging of the shower nozzle.

In Example 1, the sprinkling pipe 10 and the circulation pipe 7 used in starting up the biological desulfurization apparatus can be configured as separate lines to solve the above-mentioned clogging of shower nozzles 11. In sprinkling biologically treated water, the water is dispersed entirely via the shower nozzles 11, whereby the reaction of dissolution of hydrogen sulfide gas into the liquid and the biological reaction thereof in the carrier-packed layer can efficiently advance.

In Example 1, a microorganism-containing liquid or sludge may be any one containing sulfur-oxidizing bacteria, the habitat of which is under an environment containing hydrogen sulfide, a very small amount of oxygen and water. Specifically, the microorganism-containing liquid or sludge may be a solution treated with another operating biological desulfurization apparatus, a digestive liquid resulting from anaerobic digestive treatment, digestive sludge, or the like. The inventors have already confirmed that when a part of the surface of a digestive liquid or digestive sludge has been exposed to oxygen, the biological desulfurization apparatus can be rapidly started up with sulfur-oxidizing bacteria existing in the digestive liquid or digestive sludge.

### EXAMPLE 2

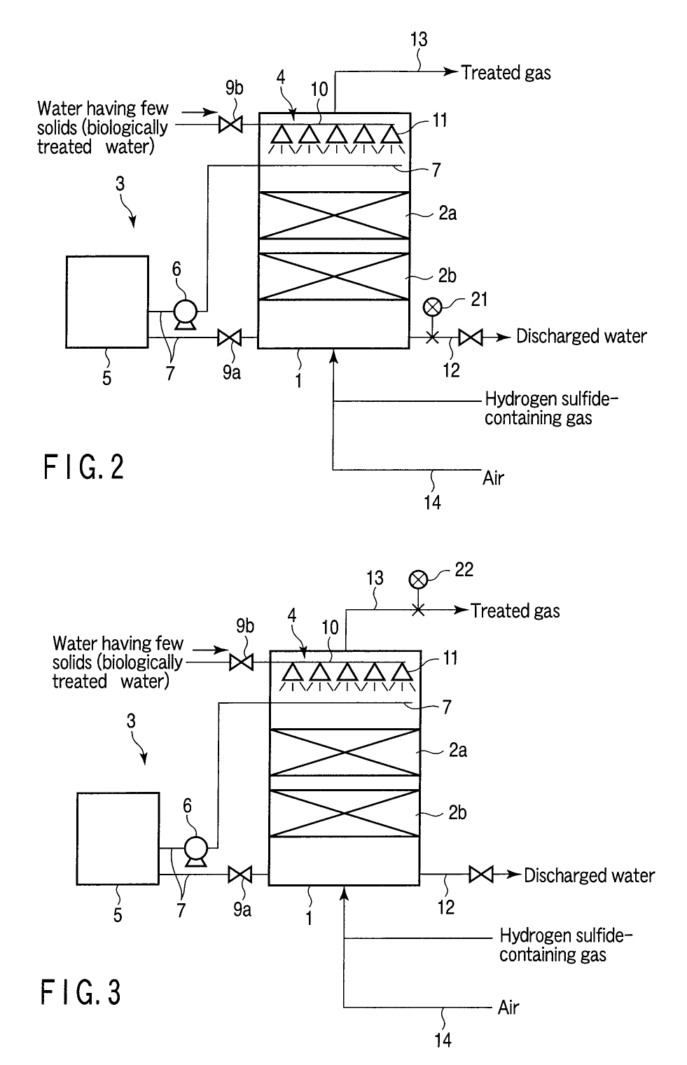

The biological desulfurization apparatus of the invention in Example 2 is described in detail with reference to FIG. 2. The same members as in FIG. 1 are assigned like numerals to omit their description, and the main part only is described.

The biological desulfurization apparatus in Example 2 is characterized in that a pH meter 21 for measuring the pH of discharged water discharged from a biological reaction tank 1 is arranged in a drain pipe 12. Alternatively, a meter for measuring alkalinity may be arranged in place of the pH meter for measuring pH.

In FIG. 2, a microorganism-containing liquid or sludge is introduced into a circulation tank 5, and the liquid or sludge is circulated simultaneously with aeration with a hydrogen sulfide gas-containing gas and an oxygen-containing gas (air), to start up the apparatus. When the sulfur-oxidizing bacteria have thereby adhered to the packed layers 2a, 2b sufficiently, hydrogen sulfide in gas is oxidized to element sulfur (S₀), a part of which is further oxidized to sulfuric acid.

When the concentration of sulfur acid is increased, the pH and alkalinity of discharged water will soon drop. With the timing of pH and alkalinity drop, circulating the microorganism-containing liquid or sludge is stopped and switched to sprinkling with biologically treated water.

The pH and alkalinity drop attributable mainly to sulfuric acid is not preferable because this drop causes corrosion of pipes in the biological desulfurization facilities and the body of the biological reaction tank 1. On the other hand, this drop serves as an indicator of sufficient adhesion of sulfur-oxidizing bacteria to the carrier-packed layers 2a, 2b.

According to Example 2, the drain pipe 12 is provided with the pH meter 21, and when pH or alkalinity lower than a predetermined level is detected, switching to sprinkling with biologically treated water is carried out thereby preventing corrosion while rapidly starting up the biological desulfurization apparatus, and even after start-up, stable treatment can be realized.

In a test conducted by the inventors, it was actually observed that when a gas containing 10000 ppm hydrogen sulfide gas was passed while a digestive liquid containing about 4000 mg/L alkali was circulated, the alkalinity dropped to about 1000 mg/L in about 3 to 4 days, followed by rapid reduction in pH (reduced from about 7 to 2-3). From this result, it is estimated that switching to sprinkling with biologically treated water is carried out preferably when the alkalinity is reduced to 1000 mg/L or less or the pH is reduced to 6 or less.

### EXAMPLE 3

The biological desulfurization apparatus of the invention in Example 3 is described in detail with reference to FIG. 3. The same members as in FIG. 1 are assigned like numerals to omit their description, and the main part only is described.

The biological desulfurization apparatus in Example 3 is characterized in that a hydrogen sulfide concentration detector 22 for measuring the concentration of hydrogen sulfide is arranged in a treated-gas pipe 13 from a biological reaction tank 1.

In FIG. 3, a microorganism-containing liquid or sludge is introduced into a circulation tank 5, and the liquid or sludge is circulated simultaneously with aeration with a hydrogen sulfide gas-containing gas and an oxygen-containing gas, to start up the apparatus. When the sulfur-oxidizing bacteria have thereby adhered to the carrier sufficiently, a part of hydrogen sulfide in the gas is oxidized to element sulfur (S₀), a part of which is further oxidized to sulfuric acid.

This is accompanied by desulfurization of hydrogen sulfide in the gas, to reduce the concentration of hydrogen sulfide in the treated gas. When the concentration of hydrogen sulfide in the treated gas has satisfied intended removal performance, it can be assumed that the carrier has a sufficient amount of organisms adhering thereto. It follows that with this timing, circulating the microorganism-containing water or sludge is stopped and switched to sprinkling with biologically treated water.

According to Example 3, the treated-gas pipe 13 is provided with the hydrogen sulfide concentration detector 22, thereby judging the timing of switching to sprinkling with biologically treated water by the concentration of hydrogen sulfide in the treated gas. Accordingly, the biological desulfurization apparatus can be rapidly started up to stabilize treatment.

In a test conducted by the inventors, it was actually observed that when a gas containing 10000 ppm hydrogen sulfide gas was passed while a digestive liquid containing about 4000 mg/L alkali was circulated, hydrogen sulfide in the treated gas was gradually decreased and in 2 to 4 days to the vicinity of 0.

### EXAMPLE 4

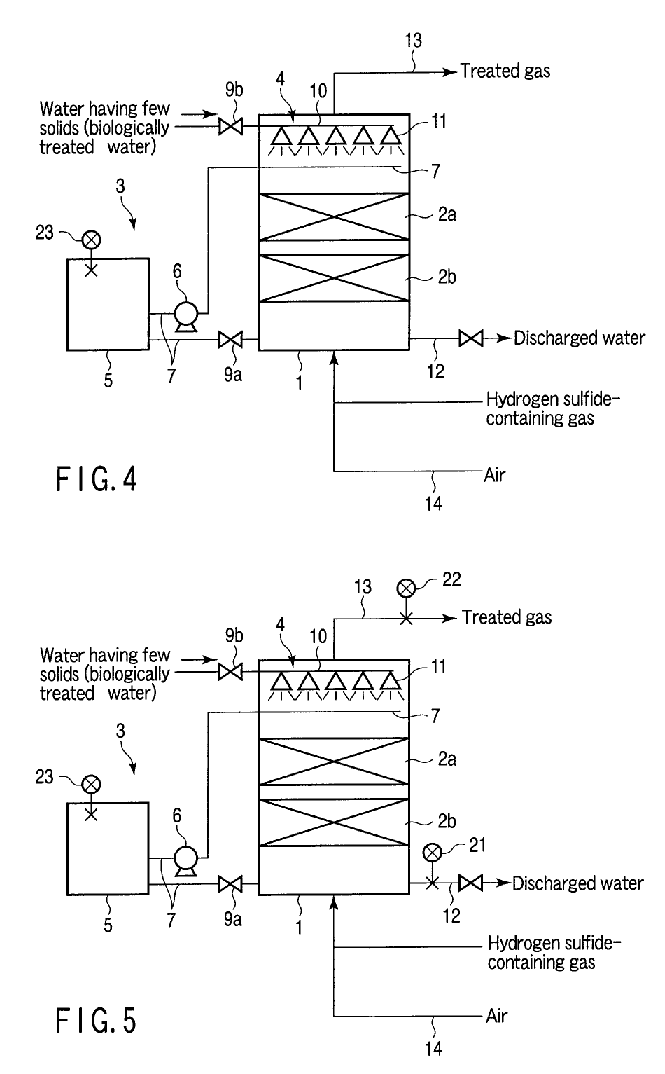

The biological desulfurization apparatus of the invention in Example 4 is described in detail with reference to FIG. 4. The same members as in FIG. 1 are assigned like numerals to omit their description, and the main part only is described.

The biological desulfurization apparatus in Example 4 is characterized in that a detector 23 (for example, a camera) for detecting the turbidity of a microorganism-containing liquid or sludge in a circulation tank 5 is arranged in the circulation tank 5. When a camera is used, the chromaticity of the sludge or liquid is judged by image analysis.

In FIG. 4, a microorganism-containing liquid or sludge is introduced into the circulation tank 5, and the liquid or sludge is circulated simultaneously with aeration with a hydrogen sulfide gas-containing gas and an oxygen-containing gas, to start up the apparatus. When the sulfur-oxidizing bacteria have thereby adhered to the carrier sufficiently, a part of hydrogen sulfide in the gas is oxidized to element sulfur (S₀), a part of which is further oxidized to sulfuric acid. As element sulfur increases, the circulating liquid turns turbid. This turbidity is detected with the detector 23 arranged in the circulation tank 5, and when the turbidity is increased to a predetermined level or more, circulating the microorganism-containing liquid or sludge is stopped and switched to sprinkling with biologically treated water.

According to Example 4, the circulation tank 5 is provided with the detector 23, thereby detecting the turbidity of the microorganism-containing liquid or sludge in the circulation tank 5, to judge an increase in element sulfur formed. By detection of this turbidity, the timing of switching to sprinkling with biologically treated water can be judged. Accordingly, the biological desulfurization apparatus can be rapidly started up to stabilize treatment.

The method of detecting turbidity in Example 4 is not limited to the detection method using a detector as described above. Examples of conceivable detection methods include visual check and a method that involves removing an aliquot of the water in the circulation tank and then judging the color of solids in the removed water by visual check or image analysis.

### EXAMPLE 5

The biological desulfurization apparatus of the invention in Example 5 is described in detail with reference to FIG. 5. The same members as in FIG. 1 to FIG. 4 are assigned like numerals to omit their description, and the main part only is described.

The biological desulfurization apparatus in Example 5 is characterized in that a pH meter 21 is arranged in a drain pipe 12, a hydrogen sulfide concentration detector 22 in a treated-gas pipe 13, and a detector 23 in a circulation tank 5.

In FIG. 5, a microorganism-containing liquid or sludge is introduced into the circulation tank 5, and the liquid or sludge is circulated simultaneously with aeration with a hydrogen sulfide gas-containing gas and an oxygen-containing gas, to start up the apparatus. When the sulfur-oxidizing bacteria have thereby adhered to the carrier sufficiently, a part of hydrogen sulfide in the gas is oxidized to element sulfur (S₀), a part of which is further oxidized to sulfuric acid. This is accompanied by an increase in element sulfur, a reduction in the concentration of hydrogen sulfide in the treated gas, and the reduction in pH and alkalinity attributable to an increase in sulfuric acid.

According to Example 5, the pH of the discharged sludge or liquid is detected by the pH meter 21, the concentration of hydrogen sulfide in the treated gas by the hydrogen sulfide concentration detector 22, and the turbidity of the liquid or sludge by the detector 23. By doing so, switching to sprinkling with biologically treated water can be judged based on the multiple items, to enable more accurate judgment.

The case where switching to sprinkling with biologically treated water was carried out based on pH, the concentration of hydrogen sulfide and the degree of turbidity has been described in Example 5 but is not restrictive, and switching to sprinkling with the water may be carried out based on two or more items, for example pH, the concentration of hydrogen sulfide, and the like.

Although the cases where a microorganism-containing liquid or sludge is used as seed sludge for biological desulfurization have been described in Examples 1 to 5 above, a digestive liquid from facilities where anaerobic digestive treatment is conducted, or digestive sludge, may be used.

1. A biological desulfurization apparatus comprising a biological reaction tank into which a hydrogen sulfide gas-containing gas is introduced, a carrier-packed layer that is arranged in the biological reaction tank and packed with a carrier for adhesion of microorganisms, a means that feeds an oxygen-containing gas to the biological reaction tank, and two or more sprinkling mechanisms that sprinkle water necessary for the organisms on the upper part of the biological reaction tank.

2. A method of starting up the biological desulfurization apparatus of claim 1, which comprises starting up the biological desulfurization apparatus while microorganisms are allowed to adhere to a carrier by feeding a hydrogen sulfide-containing gas and an oxygen-containing gas to a biological reaction tank under circulation of a microorganism-containing liquid or sludge, wherein the biological desulfurization apparatus has first and second sprinkling mechanisms wherein:
the first sprinkling mechanism is provided with a circulation tank, a circulation pump and a pipe for introducing a microorganism-containing liquid or sludge as seed sludge for biological desulfurization and circulating it through the biological reaction tank, and in the first sprinkling mechanism, the pipe for connecting the circulation tank to the biological reaction tank extends to the inside of the biological reaction tank, and the extending portion of the pipe is provided with a hole of 5 to 20 mm in diameter, and
the second sprinkling mechanism is arranged on the top of the biological reaction tank and provided with a pipe for sprinkling water on the carrier-packed layer, and the pipe in the second sprinkling mechanism extends to the inside of the biological reaction tank, and the extending portion of the pipe is provided with a nozzle excellent in an ability to disperse water.

3. The method of starting up a biological desulfurization apparatus according to claim 2, wherein the pH and/or alkalinity of discharged water from the biological reaction tank is measured, and when the pH and/or alkalinity of the discharged water from the biological reaction tank is reduced, circulating the microorganism-containing liquid or sludge is stopped and switched to sprinkling water on the upper part of the biological reaction tank.

4. The method of starting up a biological desulfurization apparatus according to claim 2, wherein the concentration of hydrogen sulfide gas in gas at the outlet of the biological reaction tank is measured, and when the concentration of hydrogen sulfide in gas at the outlet is reduced, circulating the microorganism-containing liquid or sludge is stopped and switched to sprinkling water on the upper part of the biological reaction tank.

5. The method of starting up a biological desulfurization apparatus according to claim 2, wherein the turbidity of water in the circulation tank is detected, and when turbidity in the circulation tank is confirmed, circulating the microorganism-containing liquid or sludge is stopped and switched to sprinkling water on the upper part of the biological reaction tank.

6. A method of starting up a biological desulfurization apparatus, which comprises switching from circulating the microorganism-containing liquid or sludge to sprinkling water on the upper part of the biological reaction tank by judging, by two or more of the methods of claims 3 to 5, the timing of the switching.

7. The method of starting up a biological desulfurization apparatus according to any one of claims 2 to 5, wherein a digestive liquid from facilities where anaerobic digestive treatment is conducted, or digestive sludge, is used as the seed sludge.
